(19) United States
(12) Patent Application Publication
(10) Pub. No.: US 2006/0078461 A1
(43) Pub. Date: Apr. 13, 2006

(54) SINGLE-USE AEROSOL SPRAY CAN TO DISINFECT ENCLOSED SPACES AND METHODS FOR ITS USE

(76) Inventor: Charles Kaplan, Fort Lauderdale, FL (US)

Correspondence Address: MALIN HALEY AND DIMAGGIO, PA, 1936 S ANDREWS AVENUE, FORT LAUDERDALE, FL, 33316, US

(21) Appl. No.: 11/162,970

(22) Filed: Sep. 29, 2005

Related U.S. Application Data
(60) Provisional application No. 60/522,428, filed on Sep. 30, 2004.

(51) Int. Cl. A61L 2/20

(52) U.S. Cl. 422/28; 239/333

(57) ABSTRACT

A method and apparatus for disinfecting an entire room in one operation that includes providing a sufficiently large container having a liquid disinfectant and aerosol and a manually actuated valve and nozzle that includes a spray pattern for a uniform distribution of aerosol spray, said container being sized to completely disinfect a room of a specific surface area and volume. To use the device, the aerosol container is placed at a central location in the room based on its distribution pattern and, once activated, the room will be kept closed so that the entire disinfectant contents can be continuously dispersed for total disinfectant of the room in a single operation until all the contents of the container have been emptied.

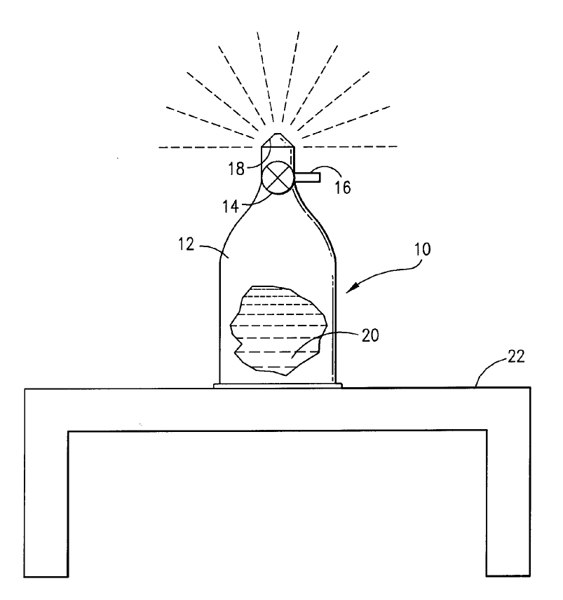

### FIELD OF THE INVENTION

This invention relates to a method for using a single-use aerosol spray can containing disinfectant to disinfect a room, building, or other enclosed space, and the article of manufacture used in this method.

### DESCRIPTION OF THE RELATED ART

Bacterial and microbial contamination of rooms and other confined spaces, such as air conditioning duct-work, has long been a source of infection for humans, particularly in dwelling areas, hospitals, classrooms, and public restrooms. Bacteria, viruses, and other disease-causing microbes adhere to surfaces after contact with humans and also linger in the air within a room after being discharged by a person sneezing or coughing. In this way, humans have spread infectious diseases among each other since time immemorial, including the common cold, influenza, rotavirus, hepatitis A, tuberculosis, conjunctivitis, staphylococcal bacterial infections, and strep throat and other streptococcal bacterial infections.

Several patents describe devices for dispensing aerosolized or pressurized liquids from a container. One such device (U.S. Pat. No. 3,137,414) proposes an aerosol can assembly and an activating cap that can be set into an operating position so that the contents of the container can be completely dispensed therefrom. Still another invention (U.S. Pat. No. 3,325,064) concerns a device designed to depress an aerosol actuator until the contents of the container are completely discharged. The prior art also describes using these aerosol containers as total release bombs for effectively fogging a room to eradicate insect infestations. U.S. Pat. No. 4,882,873. However, the prior art does not disclose any aerosol total release bombs used to disinfect a room or other enclosed space of bacteria and other microbes.

Most disinfectants are sold as liquid compositions that can be sprayed on surfaces to kill bacteria, viruses, and other microbes. However, for a person to spray an entire room with disinfectant using the current consumer-sized spray cans would be both tedious and time-consuming. Currently, no known disinfectants or disinfectant methods are efficient to disinfect a room effectively and in a reasonable amount of time.

### SUMMARY OF THE INVENTION

Accordingly, one object of this invention is to provide a single-use aerosol spray can assembly which can effectively disinfect a room or other enclosed space by killing bacteria, viruses, and other disease-causing microbes present therein using the method hereinafter described.

Another object of the invention is to provide a single-use room disinfectant and method for its use which disinfects with only minimal effort on the part of its operator.

Still another object of this invention is to provide an inexpensive and effective disinfectant and method for its use that can be employed in dwellings, schools, hospitals, and public restrooms.

The aforementioned objects of the present invention can be realized through the use of a single-use aerosol spray can assembly for disinfecting a room or other enclosed space, comprising an aerosol container fitted with a standard fogging nozzle and containing a disinfectant solution under pressure, most preferably a disinfectant for home use sold under the trademark Lysol and an industrial-grade disinfectant for hospital use, and a device designed to continuously depress the actuator valve in the open, dispense mode on the aerosol can until all of its contents under pressure have been dispensed which will be understood by one skilled in the art.

To disinfect the subject room or other enclosed space, the components of the invention described above are used as follows:

(a) position the aerosol spray container in the center of the room to be disinfected and close all sources of ventilation to the room, including doors and windows;

(b) configure the device designed to depress the container's actuator into its activated position so that the actuator is fully depressed;

(c) evacuate all people and animals from the room for a period of time sufficient to allow the contents of the aerosol container to be fully discharged; and

(d) repeat this process as needed, and more often in the case of hospitals, classrooms, and public restrooms where a great number of transient people are present and the risk for infectious germs is greater.

In accordance with these and other objects which will become apparent hereinafter, the instant invention will now be described with particular reference to the accompanying drawings.

### BRIEF DESCRIPTION OF THE DRAWINGS

The drawing illustrates a side elevational view of the invention in operation.

### DETAILED DESCRIPTION

The invention includes a method for efficiently disinfecting an entire room using a sprayed aerosol disinfectant that includes a bactericide and an aerosol container having a manually actuated valve and spray nozzle that can atomize the liquid disinfectant. The manually actuated valve can be set at an open position that allows the spray nozzle to dispense continuously the disinfectant in aerosol form in a continuous spray until the entire disinfectant contents are emptied, once the manually actuated valve is moved to the open position.

Referring now to the drawing, the invention 10 is accomplished using an aerosol container 12 that includes the disinfectant 20 in a liquid form under pressure such that when the manually actuated valve 14 is opened by lever 16, the contents 20 which is a disinfectant will be sprayed as a vapor from the nozzle 18. The actuator valve 14 is such that once the valve 14 is in the open position, the spray nozzle 18, because of the pressure inside the container which is well above atmospheric pressure, will dispense the entire contents in the surrounding room. The atomized disinfectant will be spread (in the direction of the arrows) to the surrounding wall, floor and ceiling surfaces by the air molecules in the room. The spray nozzle design will be directed to spray in a uniform hemispherical pattern surrounding the nozzle that is vertically oriented.

The volume of liquid 20 in the container 12 and the pressure will be sized relative to the container so that there is a sufficient liquid disinfectant to safely cover the surface area of a room of a predetermined size. For example, a single room that is 15 by 15 feet having four walls 7 feet high, a ceiling and a floor contains a predetermined amount of surface area. The amount of appropriate liquid in fine spray that will be spread and carried around the room by the air in the room can determine the approximate volume of liquid required.

The invention includes an article of manufacture for disinfecting a room or other enclosed space which produces a fog that is antibacterial, antiviral, and antimicrobial, and a method for its use. The article of manufacture is to be used in the following manner.

First, all sources of ventilation in the room, such as windows and doors, should be closed so as to confine and concentrate a fog produced by an aerosol spray can to one particular enclosed space. After closing the room's ventilation sources, position the aerosol spray container in the center of the room to be disinfected to achieve maximum effective dispersal of the disinfectant contents. The drawing illustrates the invention 10 placed upon a table 22 while in operation.

The aerosol spray container consists of a cylindrical container and an assembly that includes an actuator and a standard fog nozzle to disperse the container's contents over all of the surfaces in the room. In addition, the assembly includes a device designed so that, when configured by its operator into an activated position, it will maintain the container's actuator in a depressed configuration until the contents of the container have been completely discharged. The device designed to maintain the actuator in a depressed configuration is not described herein, but will be known to one skilled in the art. The container should be filled with a solution of disinfectant, preferably Lysol disinfectant for home, classroom, and nursing facility usage, and also preferably a hospital or industrial-grade disinfectant for use in hospitals and public restrooms.

The manual lever 16 is connected to the valve 14 and moved by the operator into the open valve position so that the valve is fully open. The contents of the container will begin to be discharged in an aerosolized spray or fog. All people and animals must be evacuated from the room for a period of time sufficient to allow the contents of the aerosol container to be fully discharged. As the disinfectant is dispensed from the container, it will be distributed throughout the interior of the room or enclosed space, and will settle from the air to create a coating of disinfectant microdroplets on all exposed surfaces in the room. This method is particularly effective for thoroughly disinfecting an entire room, and is more time-efficient and cost-effective than an individual using a hand-held spray can to spray every surface in a room. The entire process should be repeated as needed, and more often in the case of hospitals, classrooms, and public restrooms through which a large number of transient people pass and in which the risk of exposure to infectious germs is greater.

The instant invention has been shown and described herein in what is considered to be the most practical and preferred embodiment. It is recognized, however, that departures may be made therefrom within the scope of the invention and that obvious modifications will occur to a person skilled in the art.

What is claimed is:

1. The method of disinfecting a room to remove bacteria and microbiological contamination of the room and air conditioning ducts comprising the steps of:
(a) providing an aerosol container with a disinfectant under high pressure well above ambient pressure in a liquid form that can become a gas at ambient room temperature once the pressure is released from the aerosol container, said container having a sufficient volume of disinfectant and being sized to provide enough disinfectant to thoroughly disinfect a particular room based on the room, volume and area;
(b) locating the aerosol disinfecting container at a central strategic location in the room to ensure that the entire room area, walls, floor and ceiling are completely disinfected and receive the aerosol spray from the aerosol can; and
(c) releasing the disinfectant from the aerosol container in a desired spray pattern continuously until the entire disinfectant contents of the aerosol container are released into the room by activating a container open valve to the open position.

2. A method as in claim 1, including the additional step of:
(d) creating a spray pattern that provides a uniform spray in distribution of disinfectant throughout a specific room area and volume.

3. A method as in claim 1, including the step of:
(e) selecting a distribution valve that allows the flow of gaseous disinfectant to be emitted from the container at a pre-determined volume for a pre-determined amount of time until the disinfectant container is empty.

4. An aerosol spray container for disinfecting a room comprising:
a high pressure container capable of containing a liquid under sufficient pressure that includes an aerosol and disinfectant under high pressure;
a valve connected to said container that has a closed position and a full open position;
a nozzle for creating a vaporous spray connected to the outlet of said valve, said nozzle being sized in conjunction with the valve size and shaped to produce a uniform spray of gaseous disinfectant as the vapor exits said nozzle to have the entire room disinfected; and
means for opening said valve to a full open position that remains open, said valve having a size and shape to allow a uniform distribution of liquid that is converted to a gas to be emitted from said container for a pre-determined amount of time to uniformly disinfect a room.

5. An aerosol spray container as in claim 4, wherein:
said container is sized in volume to contain sufficient disinfectant liquid under pressure to thoroughly disinfect a room of a specific volume and wall floor and ceiling surface area.
